(19) United States
(12) Patent Application Publication
(10) Pub. No.: US 2022/0117587 A1
(43) Pub. Date: Apr. 21, 2022

(54) METHOD OF SAMPLE COLLECTION

(71) Applicant: LUCENCE LIFE SCIENCES PTE. LTD., Singapore (SG)

(72) Inventors: Min-Han Tan, Singapore (SG); Christopher Goh, Singapore (SG)

(21) Appl. No.: 17/388,440

(22) Filed: Jul. 29, 2021

(30) Foreign Application Priority Data
Jul. 29, 2020 (SG) 10202007267S

(51) Int. Cl. A61B 10/00

(52) U.S. Cl. CPC A61B 10/0051

(57) ABSTRACT

Disclosed is a method of collecting naso-oropharyngeal (NOP) sample from a subject, comprising collecting a bio-mixture comprising expectorated biofluids from two regions: a. the posterior oropharyngeal region of the throat of the subject by way of hawking, secretions of which are expectorated and spat out through the mouth; and b. the nasal and nasopharyngeal regions of the subject by way of nasal hawking, secretions of which are expectorated and spat out through the mouth. Also disclosed is a kit for collecting NOP sample from a subject according to the method of any one of the preceding claims, comprising: a sterile container for collecting the NOP sample; and a stabilizing fluid.

### FIELD OF THE INVENTION

The present invention generally relates to a method of collecting sample. In particular, the present invention relates to a method of collecting naso-oropharyngeal (NOP) sample.

### BACKGROUND

Infectious diseases have been an important contributor to human morbidity and mortality, imposing a burden on public health and global economy. Virus pandemic such as COVID-19 pandemic has led to an unprecedented global economy shutdown. Countries have been deploying diagnostics tests at scale to facilitate mass testing to identify infected persons before a new wave of infections arise. Companies with facilities which employ large numbers of employees working in close quarters are also conducting systematic re-testing to ensure the employees' well-being. While laboratory automation has made it possible to rapidly scale up the systematic processing of specimens, there are obstacles at the start of the process when specimens are collected from the individual being tested.

Conventionally, nasopharyngeal (NP) swabs are considered the gold standard for collection of viral specimens to detect viral respiratory infections using PCR. To facilitate mass testing of targeted groups of people, mobile teams of healthcare workers need to be deployed to collect specimens outside the usual clinical/laboratory settings. In many instances, these specimen collection sites are outdoors, under makeshift tentages, or mobile labs. As the reagents used for collecting the specimens are temperature-sensitive, ice boxes or refrigerators are needed on site to maintain the integrity of the reagents, and to preserve the collected specimens. NP swabs are collected by trained medical personnel in an invasive manner. The healthcare worker needs to insert the swab deep into the nostril of the patient, traversing a distance equivalent from the nostril to the outer opening of the ear. Even the most experienced healthcare worker would cause some discomfort to the patient during this process, and bleeding occurs for about 5% of cases. The swab is placed into a tube containing transport media or saline, which is in turn placed in a clean secondary container. Biosafety rules often require intensive packaging resources such as triple-bagging of the specimen. Usually, a second healthcare worker is required to assist in this process to ensure that the secondary container is not contaminated. The specimen must then be transported to the testing laboratory at 2-8° C., and arrive within 72 hours to prevent degradation.

There are several drawbacks associated with NP swabs. Firstly, false negative results are quite common in current COVID-19 tests using nasopharyngeal and nasal swabs. The sensitivity of the tests is estimated to be around 70%. As many persons with COVID-19 are asymptomatic, a person receiving the false negative result would unknowingly go on to infect others, thus perpetuating the pandemic. A more accurate and sensitive method is therefore required. Secondly, NP swabs on collection are usually transported in saline or other transport medium such as Virus transport medium (VTM) and Universal transport medium (UTM) to a laboratory for diagnostic analysis. Commercially available RNA stabilization fluids prevent degradation of nucleic acid at room temperature but either lack virucidal properties or contain guanidine thiocyanate, making them unsuitable for home-use and incompatible with automation platforms. Since the virus is still viable in the collection media, there is a risk of infection if any spillage were to occur during transit. The medical technicians must work within the confines of a biosafety cabinet, in full PPE gear to clean each layer of protective bagging as they unpack the samples. Thirdly, currently, most laboratories keep primary specimens for about 1 week before discarding them, to facilitate repeat tests if required. This takes up refrigeration space with the increased test cases. Lastly, the clinical laboratory must remain open to receive these specimens every day, including Sundays and public holidays, as the specimens would degrade if not processed. Thus, an improved collection method that preserves stability of the collected sample for at least 1 week is required.

In addition, there are a few alternative methods conventionally used to collect specimens:
1. Oral fluid swab: Patients are asked to cough deeply 3-5 times to collect any phlegm or secretions in their mouth, rub the swab on both cheeks, above and below the tongue, both gums, and on the hard palate for a total of 20 seconds to ensure the swab is saturated with oral fluid. This method is relatively less invasive than the NP swabs. Clinician-supervised self-collected oral fluid swab specimens detected 90% of cases vs clinician-collected posterior NP swab specimens which detected 79% of positive cases.
2. Posterior oropharyngeal (POP) saliva/Deep Throat saliva: Patients are asked to clear saliva from the back of the throat (cough out) into a sterile container as soon as possible after waking up, before any eating, drinking or teeth brushing. Positivity of POP saliva and NP specimen was 61.6% and 53.3% respectively, with 76% overall percent agreement between the two.
3. Straight saliva/drooling: This technique collects only oral fluids, thus excluding mucous secretions from oropharynx or lower respiratory tract (i.e. sputum). 22.9% and 22.5% of suspected SARS-CoV-2 (which is the virus that causes COVID-19) patients showed positive results using straight saliva and NP swab specimen respectively. The positive percent agreement between NP swab and straight saliva was 93.8%.

Although several studies have shown higher viral titres and accuracy of testing for saliva samples as compared to NP swabs, thereby supporting its use as a non-invasive and convenient alternative for molecular diagnostic testing of viruses, it is evident that neither the gold standard (NP swabs) nor the alternative saliva collection methods are able to detect all infections such as SARS-CoV-2 infections.

Thus, there is a need for a method of collecting nasopharyngeal specimens (which include not only saliva, but also other fluids/substance at the nasopharyngeal area), to address the disadvantages of the conventional methods as described above. There is a need for a method of collecting nasopharyngeal specimens, with the following characteristics: (1) non-invasive so that no trained medical personnel is required for the collection, (2) safe so that it does not impose a risk for transportation staff and lab technicians, (3) enables sample stability at room temperature and for long periods of time for convenient and cost-effective transport and storage, and (4) enables accurate detection with minimal false negative tests.

### SUMMARY

The present disclosure describes a methodology for collecting naso-oropharyngeal (NOP) sample from a subject, which can be used for the molecular testing of various viral pathogens. The method of the present disclosure seeks to achieve minimal invasiveness, safety, cost-effective transport and storage to spare cold chain logistics, and high detection accuracy.

In one aspect, the present disclosure refers to a method of collecting naso-oropharyngeal (NOP) sample from a subject, comprising collecting a bio-mixture comprising expectorated biofluids from two regions:

a. the posterior oropharyngeal region of the throat of the subject by way of hawking, secretions of which are expectorated and spat out through the mouth; and

b. the nasal and nasopharyngeal regions of the subject by way of nasal hawking, secretions of which are expectorated and spat out through the mouth.

Advantageously, the method as disclosed herein is non-invasive and more comfortable than the conventional NP swabs. It is also easy to administer and has wide outreach, as users can self-administer the method easily outside traditional healthcare settings by following the Instruction For Use (IFU) and/or the instruction video which may be translated to different languages (which enables deployment in resource-poor locations).

In another aspect, the present disclosure refers to a naso-oropharyngeal (NOP) sample obtained by the method disclosed herein.

In yet another aspect, the present disclosure refers to a method of generating stabilized NOP sample, wherein the method comprises adding a stabilizing fluid to the NOP sample disclosed herein, and mixing the stabilizing fluid with the NOP sample to thereby generate stabilized NOP sample.

In a further aspect, the present disclosure refers to a kit for collecting NOP sample from a subject according to the method disclosed herein, comprising:

a sterile container for collecting the NOP sample; and

a stabilizing fluid.

Advantageously, the stabilization fluid is guanidine-free and does not generate cyanide when in contact with bleach, making the kit suitable for use at locations where bleach is commonly used, such as home, schools, airports, and borders. It also enables stability of the sample collected at room temperature and for long periods of time for convenient and cost-effective transport and storage. In addition, the stabilization fluid enhances accuracy of testing by reducing false negative rates.

### BRIEF DESCRIPTION OF THE DRAWINGS

The invention will be better understood with reference to the detailed description when considered in conjunction with the non-limiting examples and the accompanying drawings, in which:

FIG. 1 illustrates the steps of the disclosed method of collecting naso-oropharyngeal (NOP) sample from a subject from the posterior oropharyngeal region of the throat and the nasal and nasopharyngeal regions of the subject.

FIG. 1A shows the forms on the kit outer box and the collection device for the subject to fill in the personal particulars.

FIG. 1B is a cross-section view of the subject, illustrating the method of collecting the NOP sample from the posterior oropharyngeal region of the throat of the subject, by tilting the subject's head back, followed by hawking, expectorating the secretions and spitting the secretions out through the mouth, into the collection device.

FIG. 1C is a cross-section view of the subject, illustrating the method of collecting the NOP sample from the nasal and nasopharyngeal regions of the subject, by way of nasal hawking, secretions of which are expectorated and spat out through the mouth, into the collection device.

FIG. 1D shows the collection device comprising a funnel and a collection tube, with a line marking indicated on the collection tube, demarking the minimal amount of NOP sample which needs to be collected by the subject, by repeating the steps in FIG. 1B and FIG. 1C until the amount of NOP sample collected is above the line marking.

FIG. 1E illustrates the action of adding the stabilization fluid to the NOP sample that has been collected in the collection device, washing down any sample residue on the funnel into the collection tube.

FIG. 1F illustrates the action of removing the funnel of the collection device and screwing a cap tightly onto the collection tube to prevent NOP sample spillage.

FIG. 1G illustrates the action of inverting the collection tube filled with NOP sample collected and the stabilization fluid for at least 5 times for proper mixing.

FIG. 1H illustrates the step of placing the collection device into a Clamshell Box, which was then placed into a biohazard bag, which was in turn placed into a resealable bag, and the resealable bag placed into the kit outer box, for further transport to a testing facility and virus testing.

FIG. 2A is a graphical representation of Ct values of Test and Control samples over 7 days at 37° C. FIG. 2B is a graphical representation of Ct values of a different set of samples over 8 days at room temperature (25° C.).

FIG. 3 illustrates the stability of clinical specimens during transit in summer and winter. 95% confidence intervals are represented by error bars. SARS-CoV-2 PCR Ct levels at both baseline (day 0) and day 8 timepoints were similar, demonstrating that delayed processing of specimens for up to 7 days in either summer or winter conditions (−20° C. to 40° C.) does not affect the diagnostic performance of the SARS-CoV-2 test. Ratio of the volume of the stabilizing fluid to the volume of the sample was 1:4.

FIG. 4 illustrates that NOP specimens have higher diagnostic utility than other specimen types.

### DETAILED DESCRIPTION

The present disclosure describes a methodology for collecting naso-oropharyngeal (NOP) sample from a subject, which can be used for the molecular testing of various viral pathogens.

In one aspect, the present disclosure refers to a method of collecting naso-oropharyngeal (NOP) sample from a subject, comprising collecting a bio-mixture comprising expectorated biofluids from two regions:

a. the posterior oropharyngeal region of the throat of the subject by way of hawking, secretions of which are expectorated and spat out through the mouth; and

b. the nasal and nasopharyngeal regions of the subject by way of nasal hawking, secretions of which are expectorated and spat out through the mouth.

As used herein, the term “hawking” refers to a forceful effort to clear or attempt to clear the throat by expelling biofluids from the throat to the mouth, and involves exhalation. In one example, the subject tilts the head back before hawking. Tilting the head back facilitates the expelling of the biofluids from the throat to the mouth; traps saliva in the valleculae so that it is not swallowed; opens the airway to improve both nasopharyngeal and oropharyngeal collection; increases airway patency and decreases airway resistance.

As used herein, the term “nasal hawking” refers to a forceful effort to clear or attempt to clear one or both of the nasal cavities and the nasopharyngeal cavity by expelling biofluids from the one or both of the nasal cavities and the nasopharyngeal cavity to the mouth, and involves inhalation. In one example, the subject inhales before nasal hawking. Inhaling facilitates the forceful effort to expel biofluids from the one or both of the nasal cavities and the nasopharyngeal cavity to the mouth. In one example, the inhalation generates a snoring sound. In another example, the subject inhales deeply before nasal hawking.

In one example, the biofluids from the posterior oropharyngeal region of the throat of the subject in step a is collected first, according to the method as disclosed herein, then the biofluids from the nasal and nasopharyngeal regions of the subject in step b is collected, according to the method as disclosed herein. In another example, the biofluids from the nasal and nasopharyngeal regions of the subject in step b is collected first, according to the method as disclosed herein, then the biofluids from the posterior oropharyngeal region of the throat of the subject in step a is collected, according to the method as disclosed herein.

In one example, when collecting the NOP sample, each of step a and step b of the method as disclosed herein is carried out once. In another example, when collecting the NOP sample, each of step a and step b of the method as disclosed herein is repeated twice. In another example, when collecting the NOP sample, each of step a and step b of the method as disclosed herein is repeated 3 times. In another example, when collecting the NOP sample, each of step a and step b of the method as disclosed herein is repeated 4 times. In another example, when collecting the NOP sample, each of step a and step b of the method as disclosed herein is repeated 5 times. In another example, when collecting the NOP sample, each of step a and step b of the method as disclosed herein is repeated 6 times. In another example, when collecting the NOP sample, each of step a and step b of the method as disclosed herein is repeated 7 times. In another example, when collecting the NOP sample, each of step a and step b of the method as disclosed herein is repeated 8 times. In another example, when collecting the NOP sample, each of step a and step b of the method as disclosed herein is repeated 9 times. In another example, when collecting the NOP sample, each of step a and step b of the method as disclosed herein is repeated 10 times.

When collecting the NOP sample, each of step a and step b of the method as disclosed herein is repeated until a volume of the NOP sample having a sufficient load of the infectious agent (such as a virus) to be detected is collected. For example, a volume of about 0.01 mL may be sufficient if the load of the infectious agent (such as a virus) in the NOP sample is sufficiently high (for example, 10 times the Limit of Detection (LoD)) to enable detection of the infectious agent (such as a virus) using the methods as described herein. Conversely, a higher volume, such as about 5 mL may be required if the load of the infectious agent (such as a virus) in the NOP sample is low (for example, 2 times the LoD). Therefore, in one example, when collecting the NOP sample, each of step a and step b of the method as disclosed herein is repeated until a volume of at least about 0.01 mL of the NOP sample is collected. In another example, each of step a and step b of the method as disclosed herein is repeated until a volume of about 0.01 mL to about 5 mL of the NOP sample is collected. In one example, the NOP sample collected has a volume of about 0.01 mL, about 0.02 mL, about 0.03 mL, about 0.04 mL, about 0.05 mL, about 0.06 mL, about 0.07 mL, about 0.08 mL, about 0.09 mL, about 1 mL, about 1.5 mL, about 2 mL, about 2.5 mL, about 3 mL, about 3.5 mL, about 4 mL, about 4.5 mL, or about 5 mL. In yet another example, each of step a and step b of the method as disclosed herein is repeated until a volume of about 1.5 mL of the NOP sample is collected. While there is no maximum volume of the NOP sample that may be collected for use in the methods disclosed herein, the volume of the NOP sample should be one that is still capable of being stabilized using the methods and kits as disclosed herein. In one example, such a volume is about 5 mL. In another example, when collecting the NOP sample, each of step a and step b of the method as disclosed herein is carried out for at least one time, until a volume of at least about 0.01 mL of the NOP sample is collected. In another example, when collecting the NOP sample, step a and step b of the method as disclosed herein are carried out sequentially, in any order, until a volume of at least about 0.01 mL of the NOP sample is collected.

In one example, the NOP sample collected is a bio-mixture which comprises salivary gland secretions, sputum, mucosal transudate, desquamated oral epithelial cells, gingival crevicular fluid, and combinations thereof.

In one example, the NOP sample collected comprises an infectious agent to be detected. In one example, the NOP sample collected comprises a virus to be detected. In one example, the virus to be detected is in the salivary gland secretions. In another example, the virus to be detected is in the sputum. In another example, the virus to be detected is in the mucosal transudate. In another example, the virus to be detected is in the desquamated oral epithelial cells. In another example, the virus to be detected is in the gingival crevicular fluid. In another example, the virus to be detected is in at least one of salivary gland secretions, sputum, mucosal transudate, desquamated oral epithelial cells, and gingival crevicular fluid and combinations thereof.

Advantageously, the sample collected by the method of the present disclosure is from two regions: the posterior oropharyngeal region of the throat, and the nasal and nasopharyngeal regions, which comprises not only saliva, but also other components as described herein which may contain virus. Thus, the method of the present disclosure maximizes the availability of viral particles in the sample collected, and offers higher accuracy of detection as compared to the existing sample collection methods, such as NP swabs (which only collects sample at nasal and nasopharyngeal regions), POP saliva/Deep Throat saliva (which only collects saliva at posterior oropharyngeal region of the throat), and straight saliva/drooling (which only collects saliva at oropharyngeal region).

In one example, the virus is an RNA virus. In another example, the RNA virus is a single-stranded RNA virus. In another example, the RNA virus is a double stranded RNA virus. In another example, the single-stranded RNA virus is a negative-strand RNA virus. In another example, the single-stranded RNA virus is a positive-strand RNA virus. In another example, the virus is a DNA virus. In another example, the DNA virus is a double-stranded DNA virus. In another example, the DNA virus is a single-stranded DNA virus. Exemplary DNA viruses include, but are not limited to human papillomaviruses, herpesviridae, herpes simplex viruses 1 and 2 (HHV-1, HHV-2), varicella-zoster virus (HHV-3), Epstein-Barr virus (HHV-4), human cytomegalovirus (HHV-5), human herpesvirus 6A, 6B, 7 and 8 (HHV-6A, HHV-6B, HHV-7, HHV-8).

In another example, the RNA virus is selected from the group consisting of: Lymphocytic choriomeningitis virus, Coronavirus, human immunodeficiency virus (HIV), Human metapneumovirus, Poliovirus, Rhinovirus, Hepatitis A, Norwalk virus, Yellow fever virus, West Nile virus, Hepatitis C virus, Dengue fever virus, Enterovirus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Human parainfluenza viruses 1-4, Influenza virus, and Hepatitis D virus.

In another example, the RNA virus is selected from the group consisting of Coronaviridae, Flaviviridae (in particular Hepacivirus and Zika virus) and Retroviridae (in particular Lentivirus).

In another example, the virus is one that causes respiratory tract infection. In another example, the virus causing respiratory tract infection is selected from the group consisting of Influenza virus, Dengue virus, and coronavirus. In another example, the coronavirus is selected from the group consisting of Severe acute respiratory syndrome coronavirus (SARS-CoV), Severe acute respiratory syndrome coronavirus 1 (SARS-CoV-1) and Severe acute respiratory syndrome coronavirus 2 (SARS-CoV-2). In another example, the virus is SARS-CoV-2.

After the NOP sample is collected, a stabilizing fluid is then added to the NOP sample to thereby generate a stabilized NOP sample. As used herein, the term “stabilizing” or grammatical variants thereof, refers to preserving the integrity of nucleic acids in the NOP sample and preventing the degradation thereof. Likewise, the term “stabilized NOP sample” refers to the NOP sample which contains integral nucleic acid which is not degraded.

In one example, after the stabilizing fluid is added to the NOP sample, the stabilizing fluid and the NOP sample are mixed. The mixing may be conducted using any means known in the art. In one example, the mixing is via inverting a container containing the stabilizing fluid and the NOP sample. In another example, the mixing is via inverting a container containing the stabilizing fluid and the NOP sample for at least 5 times. In another example, the mixing is via shaking a container containing the stabilizing fluid and the NOP sample. In another example, the mixing is via vortexing a container containing the stabilizing fluid and the NOP sample.

In one example, the ratio of the volume of the stabilizing fluid added to the volume of the NOP sample that has been collected is selected from the group consisting of 1:1, 1:2, 1:3 and 1:4. In another example, such as in Table 1 below, the ratio of the volume of the stabilizing fluid added to the volume of the NOP sample that has been collected is 1:1. In another example, such as in FIG. 3, the ratio of the volume of the stabilizing fluid added to the volume of the NOP sample that has been collected is 1:4. In one example, the volume of the stabilizing fluid is about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL or about 20 mL. In one example, the volume of the stabilizing fluid is about 2 mL. In another example, volume of the stabilizing fluid is about 3 mL.

In one example, the stabilizing fluid is virucidal. As used herein, the term “virucidal” refers to a stabilizing fluid having the capacity to or tendency to destroy or inactivate a virus, so that the virus loses its infectivity. In another example, the virucidal stabilizing fluid destroys viral proteins to inactivate virus in the NOP sample collected. In yet another example, the virucidal stabilizing fluid suppresses replication of the virus in the NOP sample collected. In another example, the virucidal stabilizing fluid is the stabilizing fluid in SAFER™ Sample kit (Lucence). In another example, the virucidal stabilizing fluid is PrimeStore® MTM (https://lhnvd.com/primestore-mtm), Copan eNat® (https://www.copangroup.com/product-ranges/enat/), or PrimeStore® ATM (https://lhnvd.com/primestore-atm).

In one example, the virucidal stabilizing fluid inactivates the virus present in the NOP sample after being mixed with the NOP sample for a period of about 5 seconds or more. In one example, the virucidal stabilizing fluid inactivates the virus present in the NOP sample after being mixed with the NOP sample for a period of about 6 seconds, about 7 seconds, about 8 seconds, about 9 seconds, about 10 seconds, about 15 seconds, about 20 seconds, about 25 seconds, about 30 seconds, about 35 seconds, about 40 seconds, about 45 seconds, about 50 seconds, about 60 seconds, or more. In another example, the virucidal stabilizing fluid inactivates the virus present in the NOP sample after being mixed with the NOP sample for a period of about 45 seconds. The quick inactivation of virus reduces the risk of viral transmission to people involved in transporting the samples, as well as reduces the risk of viral transmission to medical technicians processing the samples in the laboratory.

In another example, the stabilizing fluid is non-virucidal. As used herein, the term “non-virucidal” refers to a stabilizing fluid which does not have the capacity to or tendency to destroy or inactivate a virus. A virus upon contact with a non-virucidal stabilizing agent does not lose its infectivity. In one example, the non-virucidal stabilizing agent is PBS (saline). In another example, the non-virucidal stabilizing agent is Copan UTM® (https://www.copanusa.com/s ample-collection-transport-processing/utm-viral-transport/).

In one example, the stabilizing fluid is guanidine-free. Unlike stabilizing fluids containing guanidine, a guanidine-free stabilizing fluid as disclosed herein does not generate cyanide when in contact with disinfectants containing bleach and therefore is suitable for use at home, airports, and borders. In addition, the guanidine-free stabilizing fluid as disclosed herein does not have the risk of producing cyanide when used in conjunction with popular automated laboratory molecular diagnostics systems. In one example, the guanidine-free stabilizing fluid is the stabilizing fluid in SAFER™ Sample kit (Lucence). In another example, the guanidine-free stabilizing fluid is PrimeStore® ATM (https://lhnvd.com/primestore-atm).

To maximize the accuracy of subsequent virus detection using technologies such as PCR, and to minimise the false negative rates, the nucleic acid of the virus needs to be intact. In one example, the nucleic acid in the stabilized NOP sample is not degraded at a temperature of about −20° C. to about 40° C. In another example, the nucleic acid in the stabilized NOP sample is not degraded at room temperature of about 15 to about 30° C. In another example, the nucleic acid in the stabilized NOP sample is not degraded at about 25° C. In another example, the nucleic acid in the stabilized NOP sample is not degraded at about 30° C. In another example, the nucleic acid in the stabilized NOP sample is not degraded at about 37° C. Thus, the stabilized NOP sample collected using the method disclosed herein does not require cold-chain transport or storage. Instead, the stabilized NOP sample may be easily transported and stored at room temperature, or a temperature as high as about 40° C., without the need for scarce refrigerator space. This could greatly save the transportation and storage costs.

In one example, the nucleic acid in the stabilized NOP sample is not degraded for a period of at least about 1 day to at least about 8 days. In another example, the nucleic acid in the stabilized NOP sample is not degraded for at least 7 days. In another example, the nucleic acid in the stabilized NOP sample is not degraded for about 1 day, about 2 days, about 3 days, about 4 days, about 5 days, about 6 days, about 7 days, or about 8 days. Advantageously, in one example, the stabilizing fluid is able to stabilize viral specimens for at least 7 days, when stored at temperatures up to 37° C. In contrast, virus collected in saline, universal transport media, or other commercially available RNA stabilizers are not stable at room temperature and start degrading immediately upon collection. Thus, using the method as disclosed herein, longer transit time between sample collection and testing in the laboratory is allowed without impact on detection accuracy.

The stabilized NOP sample is delivered to facilities such as a laboratory to detect the virus contained therein. In one example, the virus in the stabilized NOP sample is detected by PCR. In one example, the viral nucleic acid is extracted prior to detection. The nucleic acid extraction may be conducted using any available nucleic acid extraction kits, such as Virus DNA/RNA Extraction Kit II VR300 (Geneaid, Taiwan), and QIAsymphony DSP Virus/Pathogen Midi Kit (Qiagen, Germany) In another example, it is not necessary to extract the viral nucleic acid prior to detection. For example, the stabilized NOP sample may be subjected to direct PCR for detecting any virus present in the sample. The PCR may be conducted using any available PCR kits, such as Fortitude ver. 2.1 (MiRXES, Singapore), Abbott Realtime SARS-CoV-2 (Abbott, USA), and Luna universal One-Step RT-qPCR kit (NEB, USA). In another example, the virus in the stabilized NOP sample is detected by sequencing.

In another aspect, the present disclosure refers to a naso-oropharyngeal (NOP) sample obtained by the method disclosed herein.

In yet another aspect, the present disclosure refers to a method of generating stabilized NOP sample, wherein the method comprises adding a stabilizing fluid to the NOP sample disclosed here, and mixing the stabilizing fluid with the NOP sample to thereby generate stabilized NOP sample. In one example of this aspect, the NOP sample disclosed herein is a NOP sample obtained by the method disclosed herein. In one example, the stabilizing fluid is virucidal. In one example, the virucidal stabilizing fluid inactivates the virus after being mixed with the NOP sample for a period of about 5 seconds or more. In one example, the virucidal stabilizing fluid inactivates the virus after being mixed with the NOP sample for a period of about 45 seconds. In another example, the stabilizing fluid is non-virucidal. In yet another example, the stabilizing fluid is guanidine-free. In one example, the ratio of the volume of the stabilizing fluid to the volume of the NOP sample that has been collected is selected from the group consisting of 1:1, 1:2, 1:3, and 1:4. In one example, the ratio is 1:1. In another example, the nucleic acid in the stabilized NOP sample is not degraded at a temperature of about −20° C. to about 40° C. In a further example, the nucleic acid in the stabilized NOP sample is not degraded for a period of at least about 1 day to at least about 8 days. In one example, the nucleic acid in the stabilized NOP sample is not degraded for at least 7 days.

In a further aspect, the present disclosure refers to a kit for collecting NOP sample from a subject according to the method as disclosed herein, comprising:

a sterile container for collecting the NOP sample; and

a stabilizing fluid.

In one example, the sterile container comprises a collection tube with a flared opening. As used herein, “flared” refers to a shape that widens progressively toward one end (in this case, opening of the container). In one example, the flared opening resembles a cone or cone-like shape, and the narrower end of the cone is integrally connected to the collection tube. Advantageously, the flared opening facilitates the flowing of the NOP sample spat out from the mouth down into the container. In addition, the flared opening surrounds and covers the subject's mouth to reduce the amount of NOP sample spilt out to the surrounding air during the spitting process.

In another example, the sterile container comprises a funnel and a collection tube, wherein the narrow end of the funnel is removably connected to the opening of the collection tube.

In one example, the kit further comprises a component selected from the group consisting of: (a) a cap for the sterile container; (b) a protective case for housing the sterile container; (c) a biohazard bag for housing (b); (d) a resealable bag for housing (b) and/or (c); (e) a box for housing (b), (c) or (d); and (f) a sealed pouch. In one example, the sterile container is provided with a cap for covering the opening of the collection tube, so as to facilitate mixing of the collected NOP sample with the stabilization fluid, and/or to prevent spillage, for example during transport to a testing facility. Any leak proof cap may be used. In one example, the cap is a screw-cap.

In one example, the kit further comprises a Clamshell Box (or protective case), into which the sterile container containing the NOP sample mixed with the stabilization fluid is placed to protect the sample. In another example, the kit further comprises a biohazard bag into which the Clamshell Box containing the sterile container is placed. In yet another example, the kit further comprises a resealable bag (such as, but not limited to, a Ziploc bag) into which the biohazard bag containing the Clamshell Box and sterile container are placed.

In one example, the kit further comprises an outer box into which all of the above described components are placed.

In one example, the kit comprises a sterile container as described herein (optionally with a cap) for collecting the NOP sample, a stabilization fluid as described herein, a Clamshell Box, a biohazard bag, and a resealable bag. In one example, the volume of the stabilization fluid provided in this kit is 2 mL.

In one example, the kit comprises a sterile container as described herein (optionally with a cap) for collecting the NOP sample, a stabilization fluid as described herein, and a sealed pouch for keeping the contents of the kit sterile before the user opens the pouch. In one example, the volume of the stabilization fluid provided in this kit is 2 mL.

As used in this application, the singular form “a,” “an,” and “the” include plural references unless the context clearly dictates otherwise. For example, the term “a primer” includes a plurality of primers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples.

### EXAMPLES

Non-limiting examples of the disclosure will be further described in greater detail by reference to specific Examples, which should not be construed as in any way limiting the scope of the disclosure.

### Example 1—Methods

Sample Collection and Processing

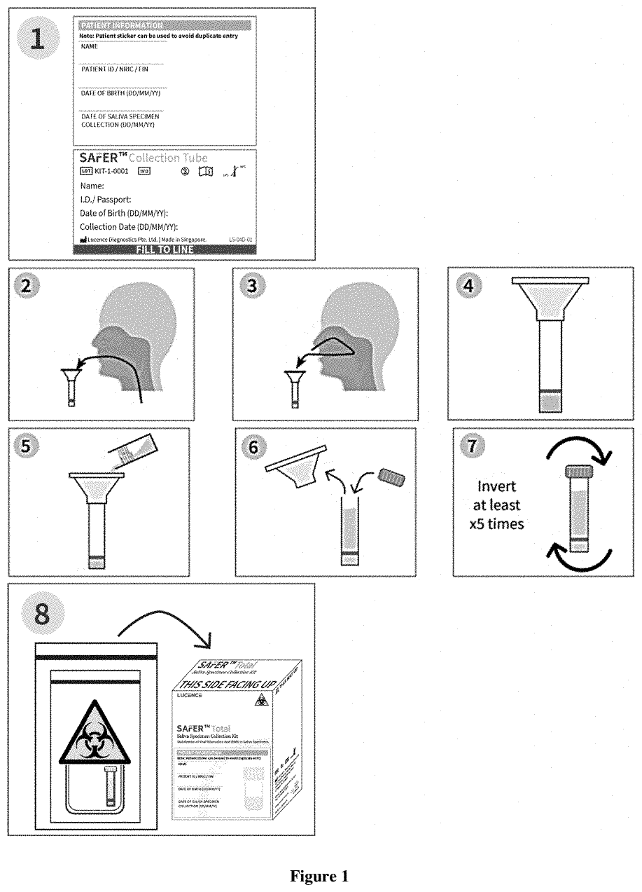

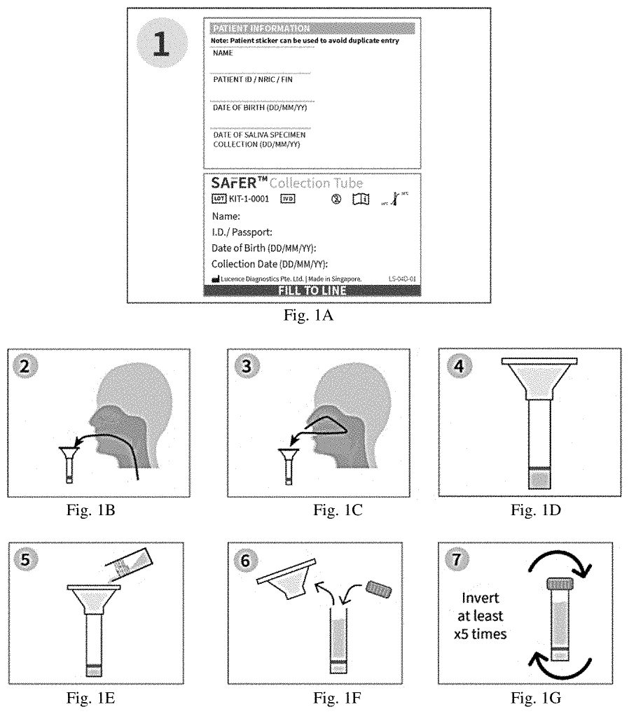

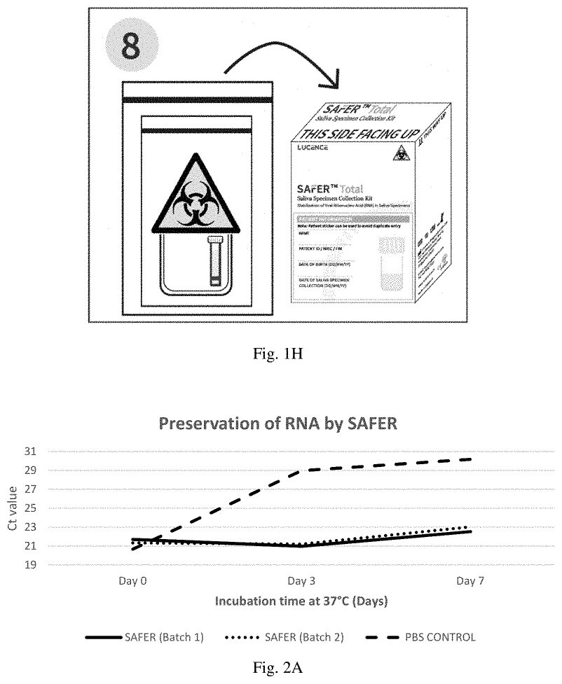

The NOP sample was collected by the steps depicted in FIG. 1A the subject's particulars were filled up on a form pasted on the container for collecting the NOP sample and on a form pasted on the resealable bag or the kit outer box used to hold the container after NOP sample was collected; (FIG. 1B) the subject's head is tilted back and hawking to clear the throat, the biofluids including saliva with sputum were expelled from the back of the throat into the mouth of the subject and spat from the mouth to a collection device (which is a collection tube connected to a funnel); (FIG. 1C) inhaling deeply and hawking the nose to clear one or both of the subject's nasal cavities, biofluids including saliva with sputum were expelled from one or both of the nasal cavities into the mouth of the subject, and spat from the mouth into the same collection device in FIG. 1B; (FIG. 1D) steps depicted in FIG. 1B and FIG. 1C were repeated until the amount of NOP sample reached a line marker on the collection tube (i.e. about 1.5 mL, bubbles do not count); (FIG. 1E) the stabilizing fluid was poured into the collection device, so that the NOP sample collected was a 1:1 mixture with the stabilizing fluid; (FIG. 1F) the funnel was removed and a cap was screwed tightly onto the collection tube to prevent spillage, and the collection tube was wiped with tissue; (FIG. 1G) the tube was gently inverted for at least 5 times for proper mixing; and (FIG. 1H) the collection tube was placed into a Clamshell Box, which was then placed into a biohazard bag and sealed, which was then put into the labelled resealable bag, and lastly into a kit outer box. The collected NOP sample was then transported to the laboratory at room temperature and stored at room temperature until further processing.

Testing the Efficacy of Stabilizing Fluid

The efficacy of SAFER™ Sample Stabilization Fluid against SARS-CoV-2 was tested in a suspension assay. Briefly, 100 μL of SARS-CoV-2 was added to each tube and incubated for the specified time (45 seconds, 5 minutes and 1 hour) at room temperature. 10-fold serial dilutions from 10⁷ log TC ID₅₀/mL were made and plated in replicates to 96-well plates containing Vero-E6 cells. Cells were incubated at 37° C. for 4 days. Cytopathic effect (CPE) was observed and virus titre calculated.

### Example 2—Results

The present disclosure describes a methodology for collecting naso-oropharyngeal (NOP) sample from a subject, comprising collecting a bio-mixture comprising expectorated biofluids from two regions: a. the posterior oropharyngeal region of the throat of the subject by way of hawking, secretions of which are expectorated and spat out through the mouth; and b. the nasal and nasopharyngeal regions of the subject by way of nasal hawking, secretions of which are expectorated and spat out through the mouth. Lucence Diagnostics' SAFER™ Sample Kit was used for the NOP sample collection. The kit comprises the SAFER™ Sample Stabilization Fluid, funnel and collection tube. The proprietary SAFER™ Sample Stabilization Fluid stabilizes nucleic acids but destroys viral proteins to inactivate viruses. In June 2020, the US FDA issued a warning letter about the dangers of guanidine thiocyanate which is found in several common commercial transport media. Cyanide gas is produced when they come into contact with bleach, rendering the transport media incompatible with some widely used laboratory testing systems. The SAFER™ Sample Kit is guanidine-free. It was demonstrated that following incubation of SAFER™ Sample Stabilization Fluid with SARS-CoV-2 virus cultured in a Biosafety Level 3 (BSL3) laboratory, the virus was no longer viable after 45 seconds (Table 1). This reduces the risk of viral transmission to people involved in transporting the samples, as well as reduces the risk of viral transmission to medical technicians processing the samples in the laboratory.

TABLE 1
SAFER ™ Sample Stabilization Fluid demonstrated
virucidal activity (>4 log reduction of viral
titre) against SARS-CoV-2 within 45 seconds.

| | Virus Titre (Log TCID₅₀/mL | | |
|---|---|---|---|
| | 45 secs (n = 3) | 5 mins (n = 1) | 1 hour (n = 4) |
| Control | 1.5 × 10⁵ ± 0 | 1.5 × 10⁵ ± 0 | 1.6 × 10⁵ ± 6.9 × 10⁴ |
| SAFER ™ | Undetectable | Undetectable | Undetectable |

Ratio of of the volume of the stabilizing fluid to the volume of the sample is 1:1

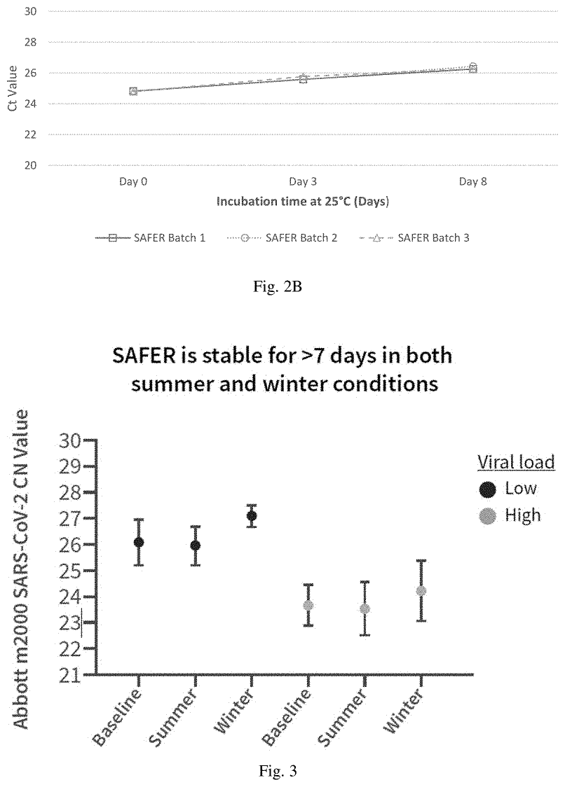

The SAFER™ Sample Stabilization Fluid has been found to stabilize viral specimens for at least 7 days, when stored at temperatures as high as 37° C. (FIG. 2). In contrast, virus collected in saline, universal transport media, or other commercially available RNA stabilizers are not stable at room temperature and start degrading immediately upon collection. As can be seen in FIG. 2A, the Ct values were consistent for SAFER™ Sample Stabilization Fluid (Batch 1 and 2) showing that the SAFER™ Sample Stabilization Fluid can preserve the integrity of the RNA throughout the 7-day period. However, an increase in Ct values of the PBS control sample was observed for Days 3 and 7 compared to Day 0, indicating that significant RNA degradation occurred in the absence of SAFER™ Sample Stabilization Fluid. FIG. 2B shows consistent stability data for three batches of samples using SAFER™ Sample Stabilization Fluid over 8 days at a temperature of 25° C.

In addition, clinical specimens stored over 8 days under summer conditions and winter conditions (with temperatures of −20° C. to 40° C.) showed similar Ct values (FIG. 3). This means that extended transit times of up to 8 days (about 1 week) between sample collection and delivery to the testing laboratory under summer and winter conditions would not affect the diagnostic performance of the SARS-CoV-2 test.

To evaluate the performance of the SAFER™ Sample Kit, paired nasopharyngeal and NOP saliva samples were collected from >100 persons, who presented themselves for Covid testing as part of an active Covid surveillance exercise. The samples were processed according to Applicant's proprietary workflow, and analysed using 3 platforms, including Applicant's lab-developed test with primers from CDC, USA and 2 commercially available Covid test kits (Fortitude ver. 2.1 (MiRXES, Singapore) and Abbott Realtime SARS-CoV-2 (Abbott, USA)) (Table 2). For each platform, results from the nasopharyngeal swabs were used as the gold-standard comparator.

TABLE 2
Performance of NOP saliva (SAFER) vs NP swabs across 3 platforms.
Overall there were 67 positive samples and 53 negative samples.

| | NP | SAFER ™ |
|---|---|---|
| Sensitivity | 56.72% | 92.54% |
| Specificity | 100.00% | 100.00% |
| No. of samples | 120 | 120 |

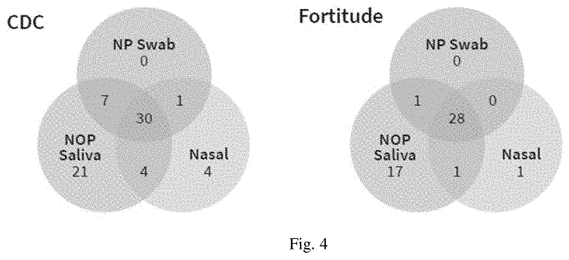

While PCR kits vary in their performance, it was observed that there is a higher diagnostic rate using NOP specimens (FIG. 4). This could be due to the observation that SARS-CoV-2 titres are higher in NOP sample containing saliva than in NP swabs. Groups in USA, China, Japan, Singapore and Malaysia have reported that saliva samples are more accurate than nasopharyngeal swabs. In FIG. 4, 120 matched patient specimens were tested using 2 different PCR kits. The number of positive diagnoses for each specimen type were shown in the Venn diagrams. As can be seen in FIG. 4, NOP sample has higher diagnostic utility than other specimen types, with 36% more positives detected, relative to NP alone. When using “CDC” PCR kit, NOP samples collected showed 62 positive diagnoses, while the total positive diagnoses of NP swab and nasal samples were 46. Thus, there was about 34.8% more positives detected using NOP sample. When using “Fortitude” PCR kit, NOP samples collected showed 47 positive diagnoses, while the total positive diagnoses of NP swab and nasal samples are 31. Thus, there were about 51.6% more positives detected using NOP sample.

In addition, the SAFER™ stabilization fluid has been validated for a variety of COVID-19 test kits (Table 3) and automation platforms and equipment (Tables 4 and 5).

TABLE 3
Third party nucleic acid extraction and PCR kits
which have been tested using clinical samples
and the SAFER ™ stabilization fluid.

| | | Manufacturer, |
|---|---|---|
| Category | Name of Kit | Country |
| Nucleic Acid | Virus DNA/RNA | Geneaid, Taiwan |
| Extraction Kit | Extraction Kit II VR300 | |
| Nucleic Acid | QIAsymphony DSP | Qiagen, Germany |
| Extraction Kit | virus/Pathogen Midi Kit | |
| SARS-CoV-2 qPCR kit | Fortitude ver. 2.1 | MiRXES, |
| | | Singapore |
| SARS-CoV-2 qPCR kit | Abbott Realtime SARS- | Abbott, USA |
| | CoV-2 | |
| SARS-CoV-2 qPCR kit | Luna universal One- | NEB, USA |
| | Step RT-qPCR kit | |

TABLE 4
Third party automated platforms which have been tested using clinical
samples and the SAFER ™ stabilization fluid.

| Name of Automated Platform | Manufacturer, Country |
|---|---|
| M2000 RealTime System | Abbott, USA |
| QiaSymphony | Qiagen, Germany |
| QIAstat-Dx | Qiagen, Germany |
| GeneXpert | Cepheid, USA |

TABLE 5
Third party PCR instrument which has been tested using clinical
samples and the SAFER ™ stabilization fluid.

| Name of Instrument | Manufacturer, Country |
|---|---|
| CFX96 Touch Real-Time PCR Detection System | Biorad, USA |

In conclusion, the SAFER™ Sample Saliva Specimen Collection Kit provides an accurate, safe, painless, non-invasive method to collect specimens for SARS-CoV-2 testing. NOP sample collected in SAFER™ Sample Saliva Specimen Collection Kit is proven to be more accurate than the gold standard nasopharyngeal swabs alone by 36%.

The virucidal properties of the SAFER™ Sample Stabilization Fluid allowed the specimen to be safely transported over long distance to a central laboratory for testing. Temperature and nucleic acid stability would reduce the number of false negatives due to reagent failure. Health authorities and payers would also enjoy cost savings in labour, logistics, and storage. SAFER™ Sample Saliva Specimen Collection Kit is thus highly suited for use in mass surveillance for COVID-19.

### Example 3—Discussion

To make the specimen collection more convenient and non-invasive, instead of the gold standard NP swabs, saliva as a specimen for SARS-CoV-2 testing is being used by Hong Kong SAR and Japan governments, including testing all passengers arriving at the airport. Similarly in the USA, Rutgers University has developed a saliva-based method and obtained FDA Emergency Use Authorization for this test. In this disclosure, the feasibility of NOP sample (a bio-mixture comprising salivary gland secretions, sputum, mucosal transudate, desquamated oral epithelial cells, gingival crevicular fluid, and combinations thereof) as a suitable diagnostic specimen for SARS-CoV-2 detection using the method as disclosed herein has been demonstrated.

The present disclosure discloses a method for NOP sample collection for optimal yield. It is demonstrated that the detection sensitivity and accuracy for SARS-CoV-2 on patient NOP specimens collected using the SAFER™ Sample Kit is highly accurate compared against nasopharyngeal swabs. These features make SAFER™ Sample Kit especially suitable for use in home-based settings, schools, at airports and borders, and mass surveillance testing.

In summary, the method as disclosed herein has the following unparalleled advantages:
1. Viral detection results based on NOP samples collected with the method as disclosed herein are more accurate than the existing gold standard of nasopharyngeal (NP) swabs. For example, 36% more positives were detected using SAFER™ Sample compared to NP swabs alone.
2. The method as disclosed herein is safer than existing methods. For example, it may inactivate viruses such as SARS-CoV-2 in as short as 45 seconds, such that biohazard risks associated with spills is eliminated. In addition, the stabilizing fluid used in the method as disclosed herein is guanidine-free and does not generate cyanide when in contact with disinfectants unlike other preservation reagents, and therefore suitable for use at home, airports, and borders.
3. The NOP sample can be self-collected, and is stable at room temperature.
4. The method as disclosed herein leads to following cost savings: (1) it does not need trained workers for NOP sample collection, leading to labour cost savings; (2) it does not require cold-chain transport, leading to transport cost savings; (3) it eliminates re-testing due to degraded specimens, as the NOP samples collected are shown to be stable for at least 7 days at a temperature as high as 40° C. In addition, weekly rest day at the laboratory is possible, as samples will not degrade if delivery to the laboratory is deferred. Moreover, the collected NOP sample may be stored at room temperature, and refrigerator space is not required for specimen storage, leading to laboratory savings.
5. The method as disclosed herein is automation friendly, as it has been proven compatible with laboratory automation platforms for mass testing, such as those described in Table 4 and Table 5 above.
6. The method as disclosed herein is a non-invasive, painless, and fearless procedure, possible to be self-administered in comfort of home/office, which eliminates the need to queue in close proximity with other potentially infected persons at testing centres. It also reduces the risk of infection through aerosols during swab collection as close contact with persons being tested can be avoided using NOP sample collection according to the method as disclosed herein.

### INDUSTRIAL APPLICABILITY

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of collecting a naso-oropharyngeal (NOP) sample from a subject, comprising collecting a bio-mixture comprising expectorated biofluids from two regions:
a. the posterior oropharyngeal region of the throat of the subject by way of hawking, secretions of which are expectorated and spat out through the mouth; and
b. the nasal and nasopharyngeal regions of the subject by way of nasal hawking, secretions of which are expectorated and spat out through the mouth.

2. The method of claim 1, wherein the bio-mixture comprises salivary gland secretions, sputum, mucosal transudate, desquamated oral epithelial cells, gingival crevicular fluid, or combinations thereof.

3. The method of claim 1, wherein the biofluids from the posterior oropharyngeal region of the throat of the subject are collected by way of tilting the subject's head back followed by hawking, expectorating the secretions, and spitting the secretions out through the mouth.

4. The method of claim 1, wherein the biofluids from the nasal and nasopharyngeal regions of the subject are collected by way of inhaling followed by nasal hawking, expectorating the secretions and spitting the secretions out through the mouth, wherein optionally the inhaling generates a snoring sound.

5. The method of claim 1, wherein the NOP sample comprises virus.

6. The method of claim 5, wherein the virus is an RNA virus.

7. The method of claim 6, wherein the RNA virus is:
(a) selected from the group consisting of: Lymphocytic choriomeningitis virus, Coronavirus, human immunodeficiency virus (HIV), Human metapneumovirus, Poliovirus, Rhinovirus, Hepatitis A, Norwalk virus, Yellow fever virus, West Nile virus, Hepatitis C virus, Dengue virus, Enterovirus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Human parainfluenza viruses 1-4, Influenza virus, and Hepatitis D virus;
wherein optionally the virus is selected from the group consisting of Influenza virus, Dengue virus, and coronavirus;
wherein optionally the coronavirus is selected from the group consisting of Severe acute respiratory syndrome coronavirus (SARS-CoV), Severe acute respiratory syndrome coronavirus 1 (SARS-CoV-1) and Severe acute respiratory syndrome coronavirus 2 (SARS-CoV-2);
wherein optionally the coronavirus is SARS-CoV-2;
or
(b) selected from the group consisting of Coronaviridae, Flaviviridae and Retroviridae.

8. The method of claim 5, wherein the virus causes a respiratory tract infection.

9. The method of claim 1, wherein the NOP sample collected has a volume of about 0.01 mL to about 5 mL;
wherein optionally the NOP sample collected has a volume of about 1.5 mL.

10. The method of claim 1 further comprising adding a stabilizing fluid to the NOP sample that has been collected and mixing the stabilizing fluid with the NOP sample to thereby generate a stabilized NOP sample.

11. The method of claim 10, wherein the stabilizing fluid is virucidal;
wherein optionally the virucidal stabilizing fluid inactivates the virus after being mixed with the NOP sample for a period of about 5 seconds or more;
wherein optionally the virucidal stabilizing fluid inactivates the virus after being mixed with the NOP sample for a period of about 45 seconds.

12. The method of claim 10, wherein the stabilizing fluid is non-virucidal.

13. The method of claim 10, wherein the stabilizing fluid is guanidine-free.

14. The method of claim 10, wherein the ratio of the volume of the stabilizing fluid to the volume of the NOP sample that has been collected is selected from the group consisting of 1:1, 1:2, 1:3, and 1:4;
wherein optionally the ratio of the volume of the stabilizing fluid to the volume of the NOP sample that has been collected is 1:1.

15. The method of claim 10, wherein the stabilized NOP sample includes a nucleic acid that is not degraded at a temperature of about −20° C. to about 40° C.

16. The method of claim 15, wherein the nucleic acid in the stabilized NOP sample is not degraded for a period of at least about 1 day to at least about 8 days;
wherein optionally the nucleic acid in the stabilized NOP sample is not degraded for at least 7 days.

17. A kit for collecting an NOP sample from a subject according to the method of claim 1, comprising:
a sterile container for collecting the NOP sample; and
a stabilizing fluid.

18. The kit of claim 17, wherein the sterile container comprises a flared opening.

19. The kit of claim 17, wherein the stabilizing fluid is:
(a) virucidal;
wherein optionally the virucidal stabilizing fluid inactivates the virus present in the NOP sample after being mixed with the NOP sample for a period of at least about 5 seconds;
wherein optionally the virucidal stabilizing fluid inactivates the virus present in the NOP sample after being mixed with the NOP sample for a period of about 45 seconds; or
(b) non-virucidal; or
(c) guanidine-free.

20. The kit of claim 17 further comprising a component selected from the group consisting of:
(a) a cap for the sterile container;
(b) a protective case for housing the sterile container;
(c) a biohazard bag for housing (b);
(d) a resealable bag for housing (b) and/or (c);
(e) a box for housing (b), (c) and/or (d); and
(f) a sealed pouch.
